(19) United States
(12) Patent Application Publication
(10) Pub. No.: US 2025/0049470 A1
(43) Pub. Date: Feb. 13, 2025

(54) SYSTEMS, DEVICES, AND METHODS FOR PREVENTING OR REDUCING LOSS OF INSUFFLATION DURING A LAPAROSCOPIC SURGICAL PROCEDURE

(71) Applicant: Standard Bariatrics, Inc, Cincinnati, OH (US)

(72) Inventors: Adam R. Dunki-Jacobs, Cincinnati, OH (US); Mark S. Ortiz, Milford, OH (US); Jonathan R. Thompson, Cincinnati, OH (US); Richard P. Nuchols, Williamsburg, OH (US); Caleb J. Hayward, Goshen, OH (US); Robert T. Means, III, Cincinnati, OH (US); Saylan J. Lukas, Cincinnati, OH (US)

(21) Appl. No.: 18/775,567

(22) Filed: Jul. 17, 2024

Related U.S. Application Data
(63) Continuation of application No. 17/364,146, filed on Jun. 30, 2021, now Pat. No. 12,064,142.
(60) Provisional application No. 63/174,065, filed on Apr. 13, 2021.
(60) Provisional application No. 63/046,153, filed on Jun. 30, 2020.

(51) Int. Cl. A61B 17/34; A61M 13/00

(52) U.S. Cl. CPC A61B 17/3474; A61B 17/3421; A61B 17/3439; A61B 2017/3443; A61B 2017/3486; A61M 13/003

(57) ABSTRACT

Systems and methods for reducing insufflation loss due to gas leakage during a laparoscopic surgical procedure are provided. The surgical procedure includes the use of a surgical instrument having an end effector and a trocar through which the surgical instrument is inserted for access to the abdominal cavity of a patient receiving laparoscopic surgery. The trocar includes a lip, a lip seal disposed within the lip, and a cannula located beneath the lip. The systems and methods reduce or prevent a loss of insufflation when using conventional systems and methods to insert the surgical instrument through a conventional trocar.

### CROSS REFERENCE TO RELATED APPLICATIONS

This application is a continuation of U.S. patent application Ser. No. 17/364,146, filed Jun. 30, 2021, which claims priority to U.S. Provisional Patent Application No. 63/174,065, filed Apr. 13, 2021, and U.S. Provisional Patent Application No. 63/046,153, filed Jun. 30, 2020, each of which is hereby incorporated herein by reference in its entirety.

### TECHNICAL FIELD

The examples herein may be directed to laparoscopic surgery, and more particularly, to a systems, devices, and methods for preventing or reducing loss of insufflation during laparoscopic surgical procedures involving the use of trocars and surgical instruments.

### BACKGROUND

Obesity is a disease that affects a significant portion of the world's population and leads to multiple chronic medical conditions and premature death from cardiovascular events and cancer. In particular, the United States has a current, and worsening obesity epidemic. The U.S. Centers for Disease Control and Prevention (CDC) reports that over 33% of the US. population is obese, with a Body Mass Index (BMI) of over 30, and another 35-40% of the US population is overweight, with a BMI of 25-30. The CDC reports that the percent of the US population being either overweight or obese by 2018 will be 75%. The CDC also reports that obesity directly costs the U.S. economy $147 billion currently, and projects that the costs will approach $315 billion by 2020.

Further, obesity has environmental, genetic and behavioral origins but is intractable to most medical and behavioral interventions. To help reduce obesity and/or facilitate weight loss, bariatric surgery may be an option for some patients that may be overweight. Typically, bariatric surgery may be an effective long-term treatment option for patients with a BMI greater than 35. Despite the 20 million patients who are eligible for weight loss surgery in the U.S., the number of procedures per year has plateaued at about 200 thousand, eliminating any public health effect of surgery.

In recent years, a popular form of bariatric surgery may include a laparoscopic vertical sleeve gastrectomy (e.g., which may remove approximately 80% of the stomach). Laparoscopic vertical sleeve gastrectomy may be a procedure that may be safer and more effective for patients eligible for weight loss surgery. In fact, it has been accepted as the surgery that should be offered to most morbidly obese patients over, for example, laparoscopic adjustable gastric banding and laparoscopic Roux-en-Y gastric bypass. As such, the surgery has been adopted by bariatric surgeons and is now the most commonly performed weight loss surgery.

Vertical sleeve gastrectomy is typically performed using standard laparoscopic or “minimally invasive” equipment. Laparoscopic surgical techniques are typically performed using a device known as a trocar or cannula, which facilitates the introduction of laparoscopic instruments into, for example, the abdominal (peritoneal) cavity of a patient. Such procedures commonly involve filling or “insufflating” the abdominal cavity with a pressurized fluid, such as carbon dioxide, to create adequate workspace between the viscera and abdominal wall. If insufflation is not properly maintained during laparoscopic surgery, the surgeon's view of the surgical area may be obstructed.

Introduction of surgical instruments into the inflated abdominal cavity without a substantial loss of insufflation gas is desirable. Such surgical instruments can include, for example, staplers, grasping instruments, cauterizing units, light sources, cameras, among other instruments. A trocar must maintain the pressure within the cavity by sealing between the trocar and the surgical instrument being used, while still allowing the surgeon to manipulate the surgical instruments. Trocars are designed to maintain a seal before the insertion of an instrument and after the removal of the instrument. As a result, many trocars provide double sealing systems. A double sealing system can include a top or proximal seal (e.g., a lip seal) used to seal around the instrument when inserted therethrough and a duckbill seal provided below the top seal for sealing the trocar housing when the instrument is not present.

Even with a double sealing system, it is expected to have some insufflation loss while the instrument is being inserted. For example, where an instrument includes an end effector extending from a sealed shaft, the end effector may not be sealed such that gas can leak out of the trocar while the end effector is passing through the seal(s). Thus, insufflation may be lost in the time between inserting the end effector and the shaft reaching the sealing system. As a result, a need currently exists for an improved trocar assembly that reduces or prevents insufflation loss while an instrument is being inserted through the trocar.

### SUMMARY

The following provides a summary of certain example implementations of the disclosed inventive subject matter. This summary is not an extensive overview and is not intended to identify key or critical aspects or elements of the disclosed inventive subject matter or to delineate its scope. However, it is to be understood that the use of indefinite articles in the language used to describe and claim the disclosed inventive subject matter is not intended in any way to limit the described inventive subject matter. Rather the use of “a” or “an” should be interpreted to mean “at least one” or “one or more”.

One implementation of the disclosed technology provides a system for reducing insufflation loss due to gas leakage during a laparoscopic surgical procedure, comprising a trocar through which a surgical stapling instrument having a stapler portion is inserted for access to the abdominal cavity of a patient receiving laparoscopic surgery, wherein the trocar comprises: a lip; a lip seal disposed within the lip; a cannula located beneath the lip, wherein the cannula includes an upper portion and a lower portion; a sealing valve disposed within the upper portion of the cannula; and a sealing device disposed within the lower portion of the cannula. The sealing device may include a balloon disposed within the lower portion of the cannula, wherein the balloon is attached to a balloon pump tube that is operative to inflate or deflate the balloon.

Another implementation of the disclosed technology provides a system for reducing insufflation loss due to gas leakage during a laparoscopic surgical procedure, comprising a trocar through which a surgical stapling instrument having a stapler portion is inserted for access to the abdominal cavity of a patient receiving laparoscopic surgery, wherein the trocar comprises a lip; a lip seal disposed within the lip; a cannula located beneath the lip, wherein the cannula includes an upper portion and a lower portion; and a diametrically expanding valve disposed within the lower portion of the cannula. The diametrically expanding valve may be a cross-slit valve, dilating valve, or trap door valve.

Another implementation of the disclosed technology provides system for reducing insufflation loss due to gas leakage during a laparoscopic surgical procedure, comprising a trocar through which a surgical stapling instrument having a stapler portion is inserted for access to the abdominal cavity of a patient receiving laparoscopic surgery, wherein the trocar comprises a lip; a lip seal disposed within the lip; a cannula located beneath the lip, wherein the cannula includes an upper portion and a lower portion; a sealing valve disposed within the upper portion of the cannula; and a sealing device, wherein the sealing device is integrated into the distal end of the stapling instrument. The sealing device may include a tip attached to an end of the stapler portion and a lip seal mounted on the tip, wherein the lip seal mounted on the tip cooperates with the lower portion of the cannula to form a seal.

Another implementation of the disclosed technology provides a system for reducing insufflation loss due to gas leakage during a laparoscopic surgical procedure, comprising a trocar through which a surgical stapling instrument having a stapler portion is inserted for access to the abdominal cavity of a patient receiving laparoscopic surgery, wherein the trocar comprises a lip; a lip seal disposed within the lip; a cannula located beneath the lip; a sealing valve disposed within the cannula; and a sealing device configured as an accessory to the trocar.

The sealing device may mount on the stapling instrument and include a hub, a retaining ring disposed within the hub, a lip seal disposed within the retaining ring, and a rigid sheath attached to the hub for covering the stapler portion of the stapling instrument. The sealing device may mount on the stapling instrument and include a hub, a retaining ring disposed within the hub, a lip seal disposed within the retaining ring, a plurality of bristles mounted in a circular pattern behind the retaining ring, and a telescoping sleeve attached to the hub for covering the stapler portion of the stapling instrument. The sealing device may mount on the stapling instrument and include a hub, a retaining ring disposed within the hub, a lip seal disposed within the retaining ring, and an unrolling sleeve attached to the hub for covering the stapler portion of the stapling instrument, wherein the sleeve includes an aperture formed in the tip thereof. The sealing device may mount on the stapling instrument and include a proximal hub, a lip seal disposed within the proximal hub, a flexible and collapsible sleeve attached to the proximal hub, and a trocar mating hub attached to the sleeve.

Another implementation of the disclosed technology provides a method for reducing insufflation loss due to gas leakage during a laparoscopic surgical procedure, wherein the surgical procedure includes the use of a stapling instrument having a stapler portion; and a trocar through which the stapling instrument is inserted for access to the abdominal cavity of a patient receiving laparoscopic surgery, the trocar comprising a lip, a lip seal disposed within the lip, and a cannula located beneath the lip, the method comprising providing a sealing device, wherein the sealing device is disposed within the cannula of the trocar, configured as an accessory to the trocar, or integrated into the stapling instrument.

The sealing device may be disposed within the cannula of the trocar and include a diametrically expanding valve. The diametrically expanding valve may be a cross-slit valve, dilating valve, or trap door valve.

The method may further comprising providing a sealing valve disposed within the cannula. The sealing device may be disposed within the cannula of the trocar and include a balloon, wherein the balloon is attached to a balloon pump tube that is operative to inflate or deflate the balloon. The sealing device may be integrated into the stapling instrument and include a blunt tip attached to an end of the stapler portion and a lip seal mounted on the blunt tip, wherein the lip seal mounted on the blunt tip cooperates with the lower portion of the cannula to form a seal. The sealing device may be configured as an accessory to the trocar that mounts on the stapling instrument and includes a hub, a retaining ring disposed within the hub, a lip seal disposed within the retaining ring, and a rigid sheath attached to the hub for covering the stapler portion of the stapling instrument. The sealing device may be configured as an accessory to the trocar that mounts on the stapling instrument and that includes a hub, a retaining ring disposed within the hub, a lip seal disposed within the retaining ring, a plurality of bristles mounted in a circular pattern behind the retaining ring, and a telescoping sleeve attached to the hub for covering the stapler portion of the stapling instrument. The sealing device may be configured as an accessory to the trocar that mounts on the stapling instrument and that includes a hub, a retaining ring disposed within the hub, a lip seal disposed within the retaining ring, and an unrolling sleeve attached to the hub for covering the stapler portion of the stapling instrument, wherein the sleeve include an aperture formed in the tip thereof. The sealing device may be configured as an accessory to the trocar that mounts on the stapling instrument and that includes a proximal hub, a lip seal disposed within the proximal hub, a flexible and collapsible sleeve attached to the proximal hub, and a trocar mating hub attached to the sleeve.

It should be appreciated that all combinations of the foregoing concepts and additional concepts discussed in greater detail below (provided such concepts are not mutually inconsistent) are contemplated as being part of the inventive subject matter disclosed herein and may be implemented to achieve the benefits as described herein. Additional features and aspects of the disclosed system, devices, and methods will become apparent to those of ordinary skill in the art upon reading and understanding the following detailed description of the example implementations. As will be appreciated by the skilled artisan, further implementations are possible without departing from the scope and spirit of what is disclosed herein. Accordingly, the drawings and associated descriptions are to be regarded as illustrative and not restrictive in nature.

### BRIEF DESCRIPTION OF THE DRAWINGS

The present disclosure will be more readily understood from a detailed description of some example embodiments taken in conjunction with the following figures:

FIG. 1 is a perspective view of a trocar assembly according to an embodiment showing the trocar.

FIG. 2 is a cross-sectional view of the trocar of FIG. 1.

FIG. 3 is a perspective view of the trocar assembly showing the trocar of FIG. 1 and an obturator.

FIG. 4 is a cross-sectional view of the trocar of FIG. 1 and the obturator of FIG. 3.

FIG. 5 is a perspective view of the trocar assembly showing the trocar of FIG. 1 and an adaptor.

FIG. 6 is a cross-sectional view of the trocar of FIG. 1 and adaptor of FIG. 5.

FIG. 7 is a bottom perspective view of the adaptor of FIG. 5.

FIG. 8 is a top perspective view of a trocar sheath according to an embodiment.

FIG. 9 is a partial cross-sectional view of the trocar sheath of FIG. 8.

FIG. 10 is a perspective view of the trocar assembly showing the trocar of FIG. 1 and the trocar sheath of FIG. 8.

FIG. 11 is a cross-sectional view of the trocar and trocar sheath of FIG. 10.

FIG. 12 is a perspective view of an example trocar showing the external components thereof.

FIG. 13 is a cross-sectional view of the trocar of FIG. 12.

FIG. 14 is a perspective view of a trocar in accordance with a first implementation of the disclosed medical devices showing the external components thereof.

FIG. 15 is a cross-sectional view of the trocar of FIG. 14.

FIG. 16 is a perspective view of the distal end of a surgical stapling instrument to which a blunt end and seal have been attached.

FIG. 17 is a perspective view of a trocar in accordance with a second implementation of the disclosed medical devices showing the external components thereof.

FIG. 18 is a cross-sectional view of the trocar of FIG. 17.

FIG. 19A depicts a hub according to an embodiment prior to the hub being placed on a stapling instrument.

FIG. 19B depicts the hub placed on the stapling instrument of FIG. 19A showing a sleeve being partially extended along the length of the end effector of the stapling instrument.

FIG. 19C depicts the sleeve of FIG. 19B fully extended along the length of the end effector such that it has reached the shaft of the stapling instrument.

FIG. 20 is a perspective view of the hub of FIGS. 19A-19C, wherein the hub includes a retaining ring, a lip seal, and a telescopic sheath or sleeve.

FIG. 21 is a perspective view of a hub according to an embodiment including a retaining ring, a lip seal, a telescopic sheath or sleeve, and a plurality of bristles mounted in a circular pattern within the hub.

FIG. 22A depicts a hub according to an embodiment prior to the hub being placed on a stapling instrument.

FIG. 22B depicts the hub placed on the stapling instrument of FIG. 19A showing a sheath being partially unrolled along the length of the end effector of the stapling instrument.

FIG. 22C depicts the sheath of FIG. 22B fully unrolled along the length of end effector such that it has reached the shaft of the stapling instrument.

FIG. 23 depicts the hub of FIGS. 22A-22C, wherein the hub includes a retaining ring, a lip seal, and a sheath having an aperture formed therein.

FIG. 24 is a side view of a collapsible sleeve assembly according to an embodiment.

FIG. 25 is a perspective view of the internal components of the proximal hub of the collapsible sleeve assembly of FIG. 24.

FIG. 26 is a perspective view of the internal components of the trocar mating hub component of the collapsible sleeve assembly of FIG. 24.

### DETAILED DESCRIPTION

Various non-limiting embodiments of the present disclosure will now be described to provide an overall understanding of the principles of the structure, function, and use of the devices, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

Trocars or abdominal access systems are available in sizes ranging from 3 mm to 12 mm (and above) and are typically divided into two main categories: cutting trocars and dilating trocars. Cutting trocars include a sharp metal or plastic blade that cuts through the various tissue layers as pressure is applied, thereby permitting easy insertion into the abdominal category. Dilating trocars include a blunt tip that separates and dilates tissue under pressure. Dilating trocars are noncutting instruments and eliminate the blade used in cutting systems for minimizing the risk of cutting internal organs.

Regardless of type, trocars typically include three main sections or components, a cannula, a lip seal, a cross-slit or intermediate check vale, and an obturator which may include a metal or plastic sharpened or non-bladed tip. The cannula is essentially a hollow tube that extends between the lip seal and the tip of the device. The lip seal is located at the top of the cannula and is intended to prevent air from escaping from the abdominal cavity while still permitting any necessary devices or equipment to be passed through the seal into the cannula. The obturator (also referred to as an awl) is located at the bottom of the cannula opposite the seal and enables the cannula to make the initial penetration into the abdomen. In addition to the lip seal, a trocar may include a one-way access valve located beneath the lip seal that permits an instrument such as a catheter or camera to open the valve when inserted into valve, but that closes upon removal of the instrument. This type of access valve may be referred to as a cross-slit valve, a duckbill valve, a flap valve, or a dome valve. A lip seal and a cross-slit valve cooperate to maintain insufflation pressure during laparoscopic surgical procedures. The lip seal maintains insufflation pressures after the shaft of a medical instrument has advanced past the seal and during device use in the surgical space. The cross-slit valve maintains insufflation when no device is present in the trocar.

Laparoscopic surgical devices having long end effectors can create leak paths in existing trocars, thereby resulting in the loss of adequate insufflation. A leak path is created when the two seals in existing trocars (e.g., the lip seal and the cross-slit valve) are both penetrated by the end portion of the laparoscopic surgical device. This leak path exists until the shaft of the surgical device reaches the lip seal which then seals around the shaft. This problem has been recognized with existing 45 mm and 60 mm stapler devices having jaws that are long enough to penetrate both seals prior to reaching the shaft of the stapling instrument. Accordingly, an accessory or device design that prevents or at least reduces air leakage during surgical device insertion past the lip seal and cross-slit valve of a trocar would be highly beneficial.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components or methods may be described solely in connection with a specific figure. Any failure to specifically describe a combination or sub-combination of components should not be understood as an indication that any combination or sub-combination is not possible. Also, for any methods described, it should be understood that unless otherwise specified or required by context, any explicit or implicit ordering of steps performed in the execution of a method does not imply that those steps must be performed in the order presented but instead may be performed in a different order or in parallel.

Example embodiments described herein can reduce the loss of insufflation when inserting an instrument through a trocar. Instruments can have an unsealed portion (e.g., end effector, such as a stapler) and a sealed portion (e.g., shaft). If the instrument being inserted has a relatively long end effector (e.g., 250 mm compared to 45 mm to 60 mm), the expected loss of insufflation when inserting the end effector through a conventional trocar could be significant. Example lengths of unsealed portions of the instrument (e.g., end effector) can be in a range of, without limitation, 60 mm to 300 mm, 65 mm to 300 mm, 100 mm to 300 mm, 100 mm to 250 mm, or 200 mm to 300 mm. Example lengths of unsealed portions of the instrument can also be greater than 60 mm or greater than 100 mm. Further, such a relatively long end effector could exit the trocar before the shaft reaches the seal(s). A substantial loss of insufflation could result in loss of visualization and, if the end effector has already exited the trocar, potential injury to the patient from inadvertent contact with patient anatomy due to the lack of visualization.

Described herein are example embodiments of apparatuses, systems, and methods for reducing insufflation loss when inserting an instrument through a trocar. In one example embodiment, a trocar assembly includes a trocar and a trocar sheath. In some embodiments, the instrument is first inserted through the trocar sheath, and the instrument and trocar sheath are then inserted together through the trocar. The trocar sheath can reduce the loss of insufflation compared to a system without the trocar sheath.

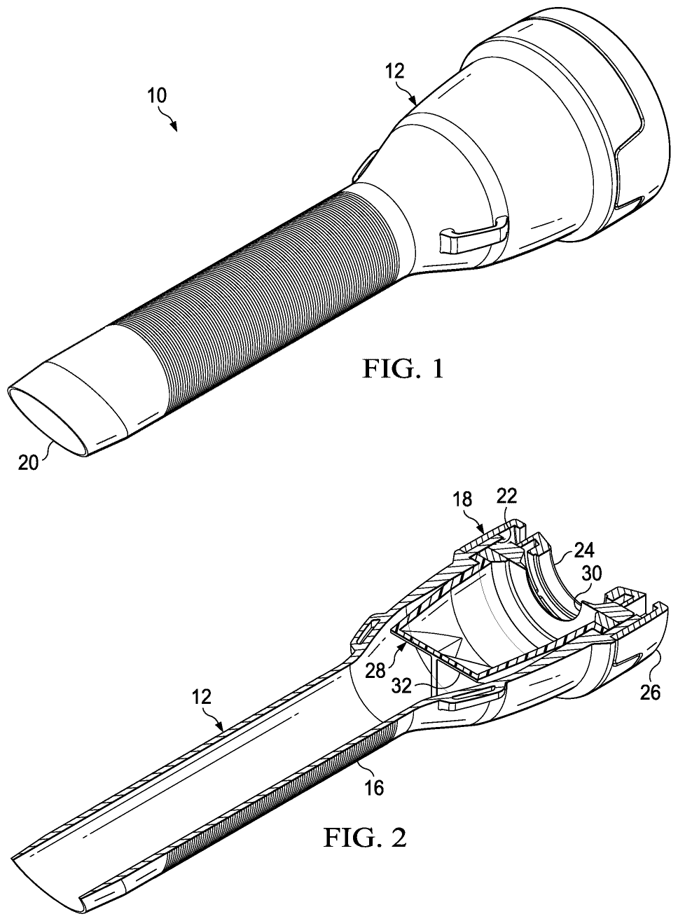

With reference to FIGS. 1-11, according to an embodiment, a trocar assembly 10 may include a trocar 12 and an obturator 14. As shown in FIG. 1, the trocar 12 can include a trocar cannula 16 and a trocar housing 18. The trocar cannula 16 defines an interior lumen (see FIG. 2) having an open distal end 20 and an open proximal end 22. The proximal end 22 extends into and is coupled to the trocar housing 18. The distal end 20 of the trocar cannula 16 can be beveled. The trocar housing 18 has an open proximal end portion that defines an opening 24. The trocar housing 18 includes cannula cap 26 and a trocar seal assembly 28. In an embodiment, the trocar seal assembly 28 includes a first seal 30 proximate the opening 24 of the trocar housing 18 and a second seal 32 distal of the first seal 30 within the trocar housing 18. In an embodiment, the first seal 30 can be a lip seal, and the second seal 32 can be a cross-slit valve or duckbill valve, such as a double duckbill valve. It will be appreciated that the trocar seal assembly can include one seal or more than two seals and have a variety of configurations. It will be recognized that the trocar seal assembly 28 cooperates with the obturator 14 or another surgical instrument extending through the trocar cannula 16 to sealingly engage the outer surface thereof and thereby preclude the passage of fluids through the trocar 12.

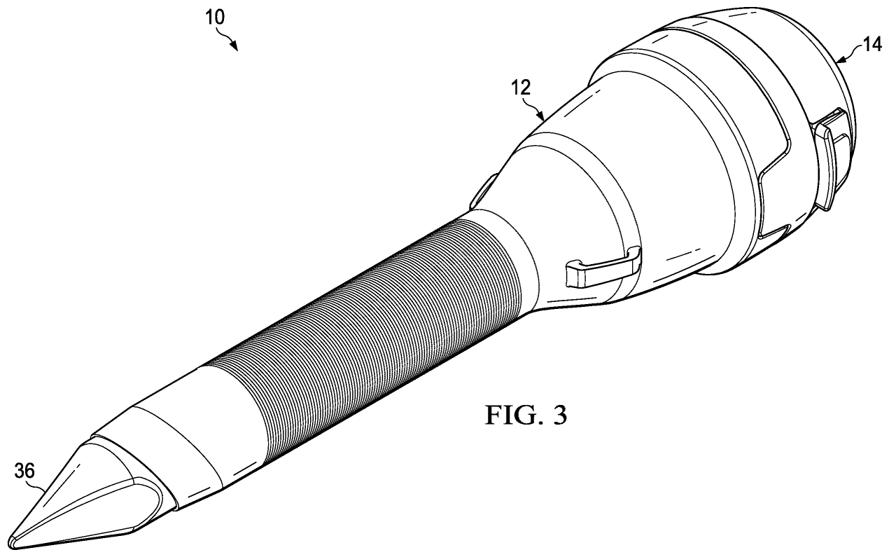

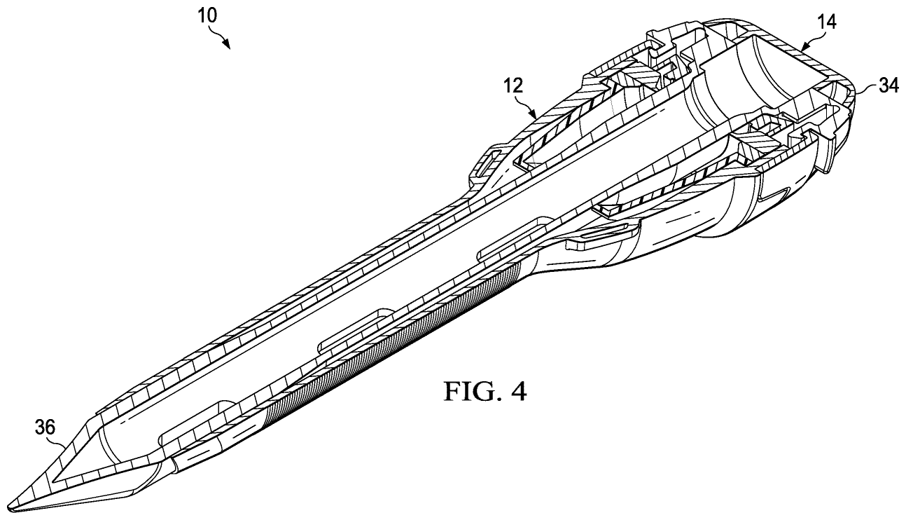

With reference to FIGS. 3 and 4, the obturator 14 is slidably and removably extendable within the trocar 12. The obturator 14 can be removably inserted into the opening 24 of the trocar housing 18, through the trocar seal assembly 28, through the trocar cannula 16 and out of the distal end 20 of the trocar cannula 16. An obturator handle 34 is provided at the proximal end of the obturator 14 and a sharpened point or blade 36 is formed at the distal end thereof. When inserted in the trocar 12, the obturator handle 34 abuts the trocar housing 18, and the blade 36 of the obturator 14 extends out of the distal end 20 of the trocar cannula 16. The trocar 12 and obturator 14 are used together to puncture a hole in soft tissue by placing the blade 36 of the obturator 14 against the tissue and pressing against the obturator handle 34. After the blade 36 breaks through the inner surface of the tissue, the obturator 14 can be removed, and the trocar 12 creates an open passageway through the tissue.

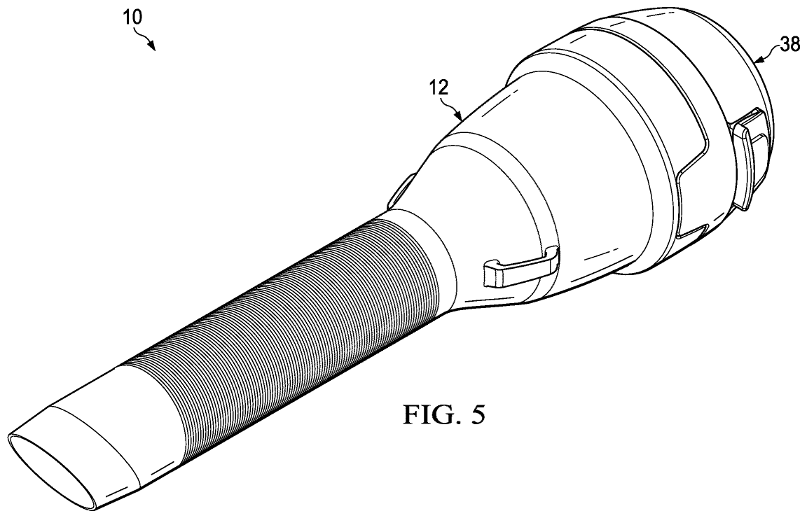

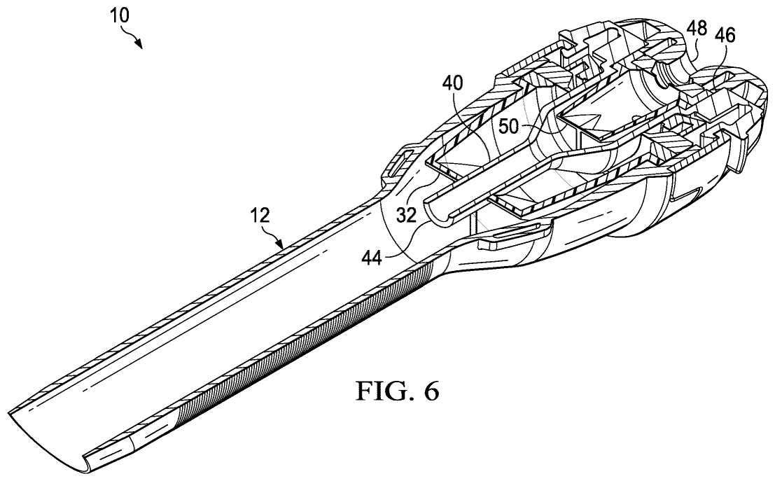

Referring now to FIGS. 5-7, in an embodiment, the trocar assembly 10 can include an adapter 38. As will be understood, the trocar 12 can be generally sized to fit instruments or tools in a particular size range. When an instrument that is significantly smaller than that size is inserted into the trocar 12, the trocar seal assembly 28 will not provide a proper seal. As shown in FIG. 5, the adapter 38 can be inserted in the opening 24 of the trocar housing 18 to allow for smaller instruments to be used with the trocar 12 while providing a proper seal. The adapter 38 includes an adapter cannula 40 extending from an adapter housing 42. The adapter cannula 40 defines an interior lumen having an open distal end 44 and an open proximal end 46. The proximal end 46 extends into and is coupled to the adapter housing 42. The adapter housing 42 has an open proximal end portion that defines an opening 48. As shown in FIG. 6, when inserted in the trocar 12, the adapter housing 42 abuts the trocar housing 18, and the distal end 44 of the adapter cannula 40 extends into and is fluidically coupled with the trocar cannula 16. The opening 48 of the adapter 38 is smaller than the opening 24 of the trocar housing 18. The adapter 38 can also include an adapter seal assembly 50. In an embodiment, the adapter seal assembly 50 can include a first seal proximate the opening 48 of the adapter housing 42 and a second seal distal of the first seal within the adapter housing 42. In an embodiment, the first seal can be a lip seal, and the second seal can be a cross-slit valve or duckbill valve, such as a double duckbill valve. It will be appreciated that the adapter seal assembly can include one seal or more than two seals and have a variety of configurations. It will be recognized that the adapter seal assembly 50 cooperates with a surgical instrument extending through the adapter cannula 40 to sealingly engage the outer surface thereof and thereby preclude the passage of fluids through the trocar 12 and the adapter 38.

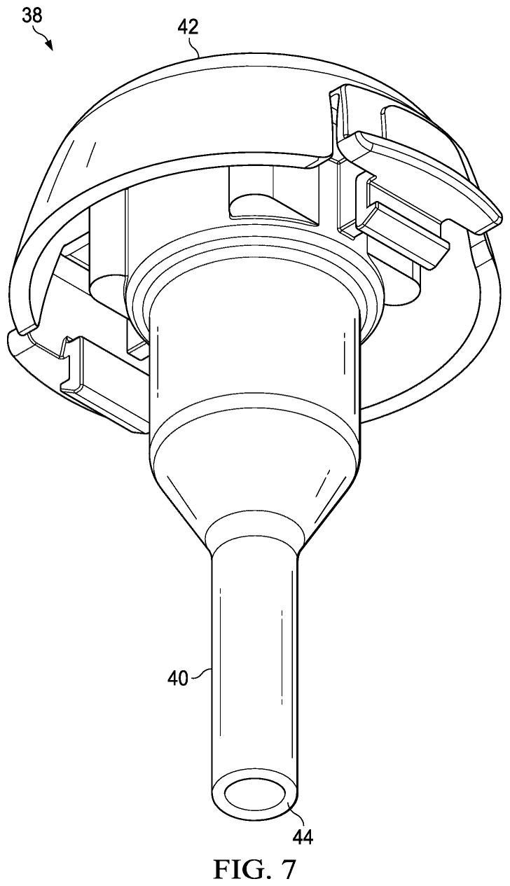

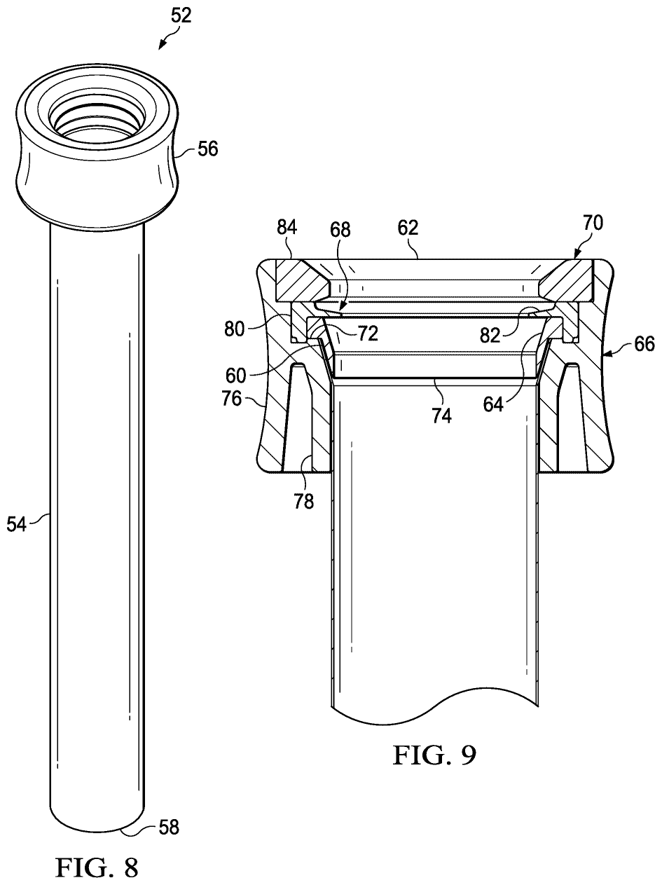

As shown in FIGS. 8-11, in an embodiment, the trocar assembly 10 can include a trocar sheath 52. The trocar sheath 52 is slidably and removably extendable within the trocar 12. The trocar sheath 52 can be removably inserted into the opening 24 of the trocar housing 18, through the trocar seal assembly 28, and through the trocar cannula 16. The trocar sheath 52 can include a sheath cannula 54 and a housing assembly 56. The sheath cannula 54 defines an interior lumen having an open distal end 58 and an open proximal end 60. The proximal end 60 extends into and is coupled to the housing assembly 56. The housing assembly 56 has an open proximal end portion that defines an opening 62. In some embodiments, the sheath cannula 54 is a thin-walled tube made from plastic, such as HDPE, that is rigid enough to not collapse during use while thin enough to not increase the required inner diameter of the trocar 12 significantly. In another embodiment, the sheath cannula 54 can be flexible. The wall thickness of the sheath cannula 54 can be in a range of, for example, 0.005 inches to 0.01 inches, such as 0.008 inches. The sheath cannula 54 can be transparent, semi-transparent, translucent, or opaque in various embodiments. While the trocar sheath 52 is shown as being a separate component from the trocar 12, in other embodiments, the trocar 12 and trocar sheath 52 may be co-molded or co-formed.

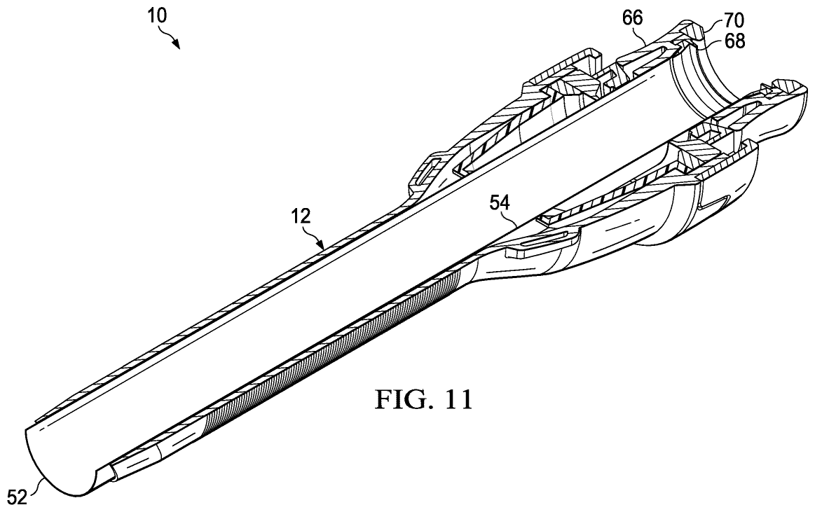

With further reference to FIGS. 9 and 11, in an embodiment, the housing assembly 56 can include a seal plate 64, a sheath hub 66, a sheath seal 68, and a sheath cap 70. The seal plate 64 may be coupled to the proximal end 60 of the sheath cannula 54. The seal plate 64 defines an opening and includes a proximal lip 72 extending from a distal skirt 74. The skirt 74 can be sized to extend into the open proximal end 60 of the sheath cannula 54. The sheath hub 66 is coupled to an exterior of the proximal end 60 of the sheath cannula 54. The proximal end 60 of the sheath cannula 54 that is captured in the sheath hub 66 can be outwardly flared to provide a mechanical locking feature without reducing the effective inner diameter of the sheath cannula 54. The seal plate 64 can be correspondingly flared. The seal plate 64 captures the sheath cannula 54 to withstand tensile loading during use.

Still referring to FIGS. 9 and 11, in an embodiment, the sheath hub 66 can include an outer wall 76 defining an opening therethrough. The outer wall can be, for example, concave and act as a grip. The sheath hub 66 can also in include an inner skirt 78 extending from the outer wall 76 towards a distal end of the sheath hub 66. Thus, the diameter of the sheath hub 66 may vary. For example, the diameter of the opening at the proximal end of the sheath hub 66 at the outer wall 76 can be larger than the diameter of the opening at the distal end of the sheath hub 66 at the inner skirt 78. It will be recognized that the configuration of the sheath hub 66 may vary. For example, the sheath hub 66 may have a single skirt wall. The sheath seal 68 and the sheath cap 70 are sized to fit in the opening of the sheath hub 66. The sheath seal 68 and the sheath cap 70 may be contained by the sheath hub 66. The sheath seal 68 includes a generally circular outer wall 80 defining an opening. In an example configuration, the sheath seal 68 is a lip seal and includes a lip 82 extending into the opening from the outer wall 80. The sheath seal 68 can be configured to have less drag relative to the shaft of the instrument inserted therethrough versus the drag between the trocar seal assembly 28 and the trocar sheath 52. In an embodiment, the drag on an instrument while it is being inserted through the sheath seal 68 may be less than the drag on the instrument while removing it through the sheath seal 68. The sheath cap 70 also includes a generally circular outer wall 84 defining an opening. The sheath cap 70 can provide, in some embodiments, axial preload on the proximal end 60 of the sheath cannula 54, the sheath seal 68, and the seal plate 64 contained in the sheath hub 66 to provide a hermetic seal to prevent air leakage in use. In various embodiments, the sheath cap 70 can be held in place using an ultrasonic welding process, glue, or a snap fit.

In an embodiment, the trocar sheath 52 may include a housing assembly 56 without a seal. The difference between the inner diameter of the sheath cannula 54 and the outer diameter of the instrument may be very small. For example, the difference between the inner diameter of the sheath cannula 54 and the outer diameter of the instrument can be in a range of 0.001 inches to 0.01 inches, 0.001 inches to 0.005 inches, 0.005 inches to 0.01 inches, or 0.002 inches to 0.005 inches.

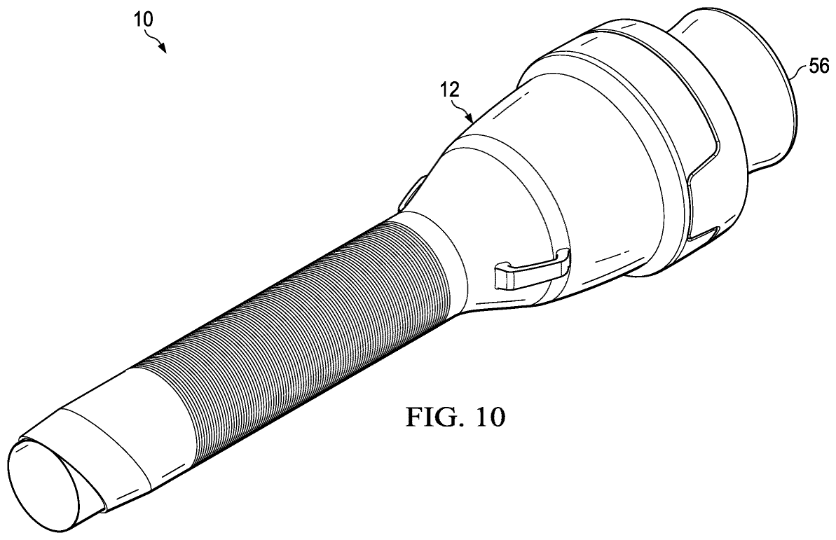

FIGS. 10 and 11 depict the trocar sheath 52 positioned in the trocar 12. As shown, the sheath cannula 54 can extend beyond the distal end 20 of the trocar cannula 16. In another embodiment, the sheath cannula 54 is sized to extend no further than the beveled end of distal end 20 of the trocar cannula 16. In other words, in such a configuration, a portion of the sheath cannula 54 can be uncovered by the trocar cannula due to the beveling, but the beveled end of the trocar cannula 16 extends past the distal end 58 of the sheath cannula 54. When the trocar sheath 52 is positioned in the trocar 12, the trocar seal assembly 28 cooperates with trocar sheath 52 to sealingly engage the outer surface thereof. Similarly, when an instrument is positioned in the trocar sheath 52, the housing assembly 56 of the trocar sheath 52 cooperates with the instrument to sealingly engage the outer surface thereof. Thus, in a configuration, the trocar 12, the trocar sheath 52, and an instrument inserted through the trocar sheath 52 cooperate to preclude the passage of fluids through the trocar 12.

In various embodiments, the trocar sheath 52 can include a locking feature on (e.g., on the sheath hub 66) to lock the proximal end of the instrument shaft (e.g., the handle of a stapler) to the trocar sheath 52. Example configurations include a quick-connect fitting, radial compression spring that locks onto the shaft; threads on sheath that snap past prongs/flanges in instrument shaft shrouds; extrusion on instrument shaft shrouds that tube slides over; undercut on handle shrouds that tube slides into; grooves on outer tube that proximal hub on sheath snaps into; or combinations thereof.

In various embodiments, a trocar sheath may be configured to be used with an existing trocar (e.g., in a retrofit manner) or a trocar designed to be used with the trocar sheath.

In use, a surgeon or other user may insert the trocar 12 and obturator 14 into the tissue of a patient. After the blade 36 of the obturator 14 breaks through the tissue, the obturator 14 can be removed from the trocar 12. If a relatively small instrument is being used, the adapter 38 can be inserted. Otherwise, the instrument to be used can be inserted into the trocar sheath 52. The distal end of the instrument is inserted into the opening 24 of the trocar housing 18. When the distal end of the instrument extends through the second seal 32, a leak path is created through the second seal 32 and the unsealed distal portion of the instrument (e.g., the end effector of the instrument). Without the trocar sheath 52, the leak path would exist as long as the unsealed portion of the instrument extends through both the first seal 30 and the second seal 32 (i.e., until the sealed portion of the instrument reaches the first seal 30). With the trocar sheath, the leak path only exists from when the distal portion of the instrument passes through the second seal 32 and when the sheath cannula 54 reaches the first seal 30. Thus, the trocar sheath 52 reduces the amount of time that insufflation could be lost while the instrument is being inserted in the trocar 12. This reduction in time would be greater for instruments with longer unsealed portions (e.g., longer staplers). For some instruments, the trocar sheath 52 seals the trocar 12 before the end effector has exited the distal end 20 of the trocar cannula 16. This reduces or eliminates the likelihood of a loss of visualization due to insufflation loss while the end effector extends out of the trocar 12. To remove the instrument from the trocar 12, the trocar sheath 52 is removed with the instrument. If the instrument is removed first and the seal between the housing assembly 56 of the trocar sheath 52 is lost, insufflation loss would likely occur as long as the sheath cannula 54 extends through the second seal 32.

As previously stated, laparoscopic surgical devices having long end effectors can create leak paths in existing trocars, thereby resulting in the loss of adequate insufflation. A leak path is created when the two seals in existing trocars (i.e., the lip seal and the cross-slit valve) are both penetrated by the end portion of the laparoscopic surgical device. This leak path exists until the shaft of the surgical device reaches the lip seal. This problem has been recognized with existing 45 mm and 60 mm stapling devices having jaws that are long enough to penetrate both seals prior to reaching the shaft of the stapler.

The jaws of some stapling devices, such as the TITAN SGS from Standard Bariatrics, Inc., are approximately 250 mm long, thereby presenting two additional problems relating to insufflation leakage. First, the time required to insert the 250 mm long jaw of the TITAN SGS stapler is 4 to 5.5 times longer than what is required for existing 45 mm and 60 mm devices, which allows more air to leak from the insufflated volume prior to reaching the shaft portion of the inserted stapling instrument. If a significant volume of air is lost in the process of inserting the stapling instrument, visualization can be lost, leading to potential patient injury from inadvertent contact with patient anatomy during the period when visualization is not possible. Second, the length of the 250 mm jaw of the TITAN SGS device is great enough to exit the trocar in the intra-abdominal cavity prior to reaching the stapling instrument shaft and stopping insufflation air leakage. This is significant because if the surgeon were to insert the stapling instrument more quickly to minimize the volume of air leakage, the stapling instrument may exit the trocar in the intra-abdominal space in an uncontrolled manner, thereby leading to potential patient injury from inadvertent contact with patient anatomy.

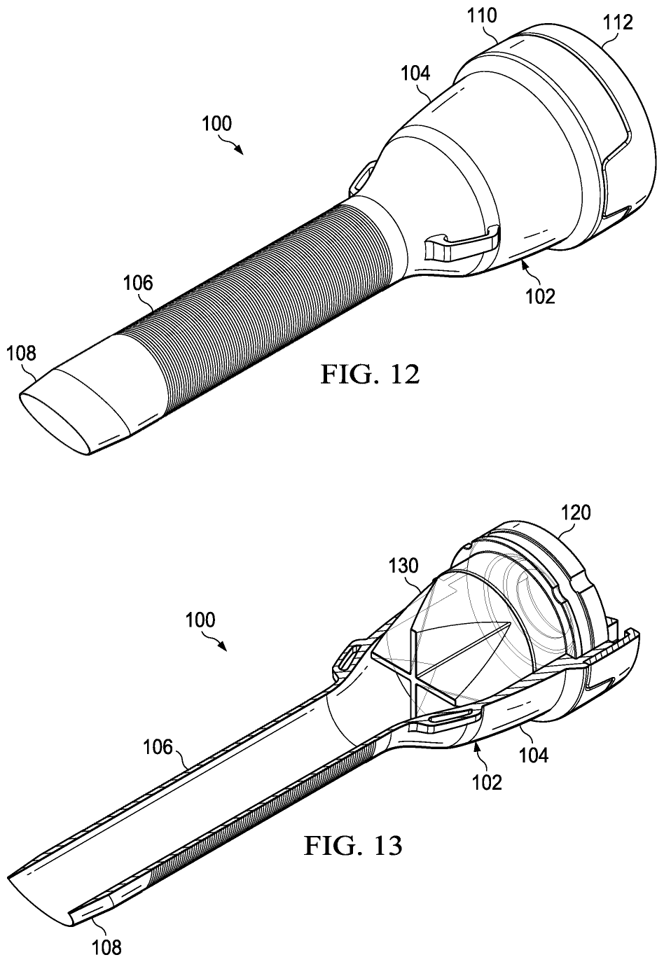

FIG. 12 provides a perspective view of example trocar 100, which includes cannula 102, lip 110 and cannula cap 112. Cannula 102 includes a tapered upper portion 104 and a tubular lower portion 106, wherein the internal diameter of upper portion 104 is greater than the internal diameter of lower portion 106, which terminates at angled tip 108. As previously described, a trocar such as that shown in FIG. 12 typically includes a seal and a valve that cooperate to maintain insufflation pressure during laparoscopic surgical procedures. The seal is typically a lip seal and the valve is a cross-slit valve. The lip seal maintains insufflation pressures after the shaft of a medical instrument has advanced past the seal and during instrument use in the surgical space. The cross-slit valve maintains insufflation when no instrument is present in the trocar. FIG. 13 provides a cross-sectional view of trocar 100, wherein lip seal 120 is visible underneath cannula cap 112 and cross-slit valve 130 is disposed within tapered upper portion 104 of cannula 102. The various implementations described below prevent air leakage during instrument insertion past lip seal 120 and cross-slit valve 130. Although cross-slit valve 130 is shown in the Figures, this valve may be unnecessary in implementations in which another valve disposed within the distal portion of the cannula prevents air leakage when the medical instrument is present in the cannula.

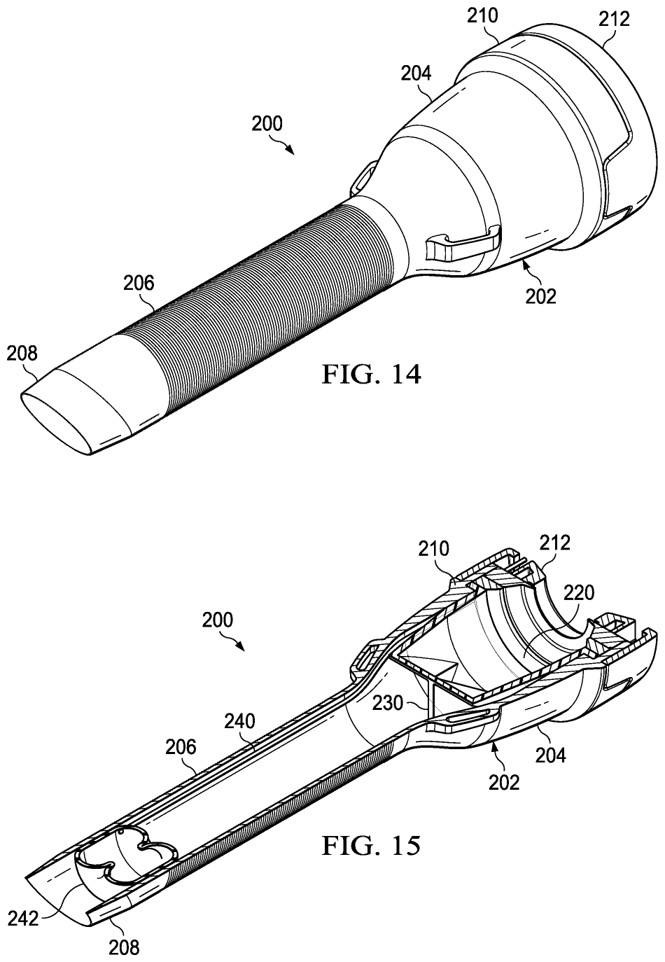

FIGS. 14 and 15 depict a distal balloon implementation, which minimizes air leakage during insertion of an instrument into a trocar when the cross-slit valve is opened and the shaft of the instrument has not reached the lip seal. FIG. 14 provides a perspective view of example trocar 200, which includes cannula 202, lip 210 and cannula cap 212. Cannula 202 includes a tapered upper portion 204 and a tubular lower portion 206, wherein the internal diameter of upper portion 204 is greater than the internal diameter of lower portion 206, which terminates at angled tip 208. FIG. 15 provides a cross-sectional view of trocar 200, wherein lip seal 220 is visible underneath cannula cap 212 and proximal cross-slit valve 230 is disposed within tapered upper portion 204 of cannula 202. Balloon pump tube 240, which inflates and deflates distal balloon 242, enters trocar 200 through cannula cap 212 and extends downward into tubular lower portion 206 of cannula 202 running parallel to the central axis of cannula 202. In this implementation, lip seal 220 is identical to that found in existing trocars and is designed to form a seal on the shaft of an instrument being inserted into the trocar 200. Proximal cross-slit valve 230 is also identical to that found in existing trocars and is designed to prevent air leaks when closed. Cannula 202 is also identical to that found in existing trocars and is designed to be a rigid tube which allows an instrument to be inserted through the tube for accessing the abdominal cavity. Cannula cap 212 is also identical to that found in existing trocars and is designed to retain lip seal 200 and proximal cross-slit valve 230 within cannula 202. Cannula cap 212 may also mate with other tools such as, for example, an obturator and a 5 mm adapter. Balloon pump tube 240 allows air to be pumped into and out of distal balloon 242, which is donut shaped and is located inside trocar 200 toward the distal end of the trocar. An instrument is pushed through the center of balloon 242, which deflates when the instrument is fully inserted. Alternative implementations include replacing the balloon with a self-inflating memory foam equivalent which would not require a balloon pump in order to inflate.

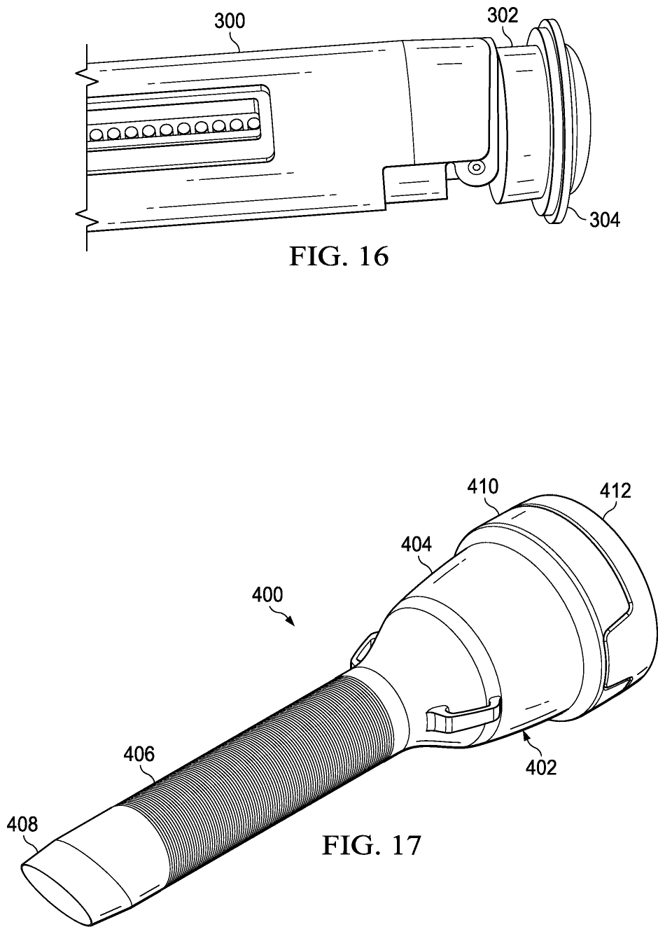

FIG. 16 depicts a stapler distal seal implementation, which is a TITAN SGS surgical stapler accessory designed to minimize air leakage during insertion of the stapling instrument when the cross-slit valve is opened and the shaft of the instrument has not reached the trocar lip seal. As shown in FIG. 16, end effector 300 includes a pin at the distal end thereof which is used to pin blunt tip 302 to the end of the end effector. End effector lip seal 304 mates to the outer diameter of blunt tip 302 and fills the air gap between the outer diameter of blunt tip 302 and the inner diameter of the trocar cannula. Blunt tip 302 combined with end effector lip seal 304 permits the distal end of end effector 300 to be inserted all the way to the distal end of the trocar before air leakage occurs, thereby minimizing the time between when air leakage begins and the shaft of the stapling instrument reaches the trocar lip seal. In some implementations, this accessory is removable from end effector 300.

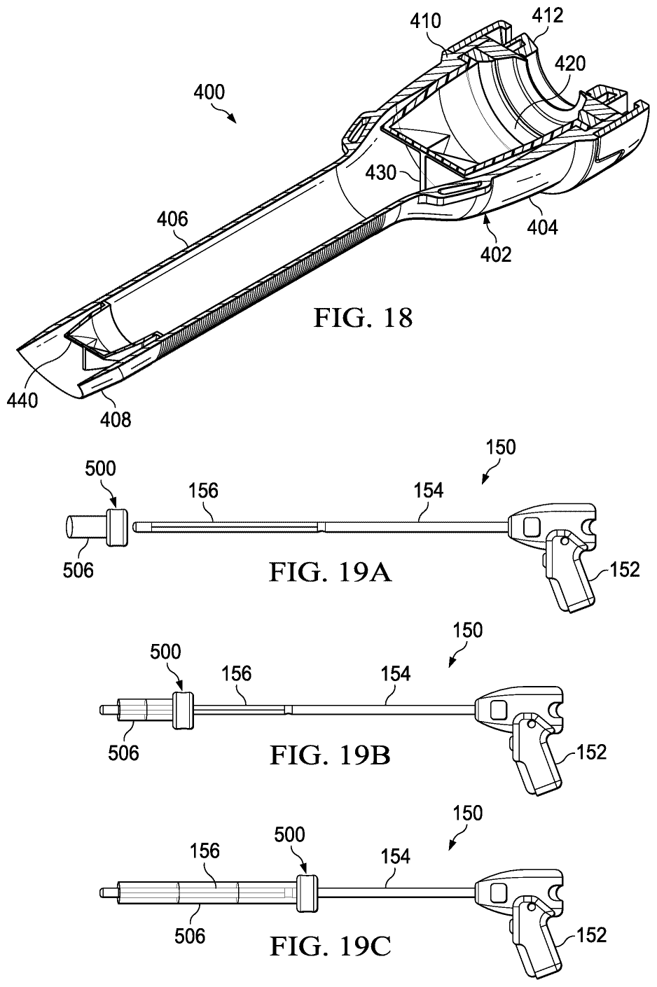

FIGS. 17 and 18 depict a distal seal implementation, which minimizes air leakage during insertion of an instrument into a trocar when the cross-slit valve is opened and the shaft of the instrument has not reached the lip seal. FIG. 17 provides a perspective view of example trocar 400, which includes cannula 402, lip 410 and cannula cap 412. Cannula 402 includes a tapered upper portion 404 and a tubular lower portion 406, wherein the internal diameter of upper portion 404 is greater than the internal diameter of lower portion 406, which terminates at tip 408. FIG. 15 provides a cross-sectional view of trocar 400, wherein lip seal 420 is visible underneath cannula cap 412 and proximal cross-slit valve 430 is disposed within tapered upper portion 404 of cannula 402. In various implementations, valve 430 is absent from cannula 402 as it is not necessary. Distal valve 440 is disposed within the distal portion of tubular lower portion 406. In this implementation, lip seal 420 is identical to that found in existing trocars and is designed to form a seal on the shaft of an instrument being inserted into the trocar 400. Proximal cross-slit valve 430 is also identical to that found in existing trocars and is designed to prevent air leaks when closed. Cannula 402 is also identical to that found in existing trocars and is designed to be a rigid tube which allows an instrument to be inserted through the tube for accessing the abdominal cavity. Cannula cap 412 is also identical to that found in existing trocars and is designed to retain lip seal 420 and proximal cross-slit valve 430 within cannula 402. Cannula cap 412 may also mate with other tools such as, for example, an obturator and a 5 mm adapter. Distal valve 440 prevents air leakage when closed. By placing valve 440 toward the distal end of trocar 400, an instrument can be inserted further into trocar cannula 402 before opening the valve and permitting air leakage, thereby minimizing the time between when the air leakage begins, and the shaft of the instrument reaches the lip seal. Distal valve 440 may be a cross-slit valve, trap door valve, dilating valve, or the like. One alternate implementation does not include proximal cross-slit valve 430 and another alternate implementation locates distal valve 440 entirely at the distal opening of trocar 400.

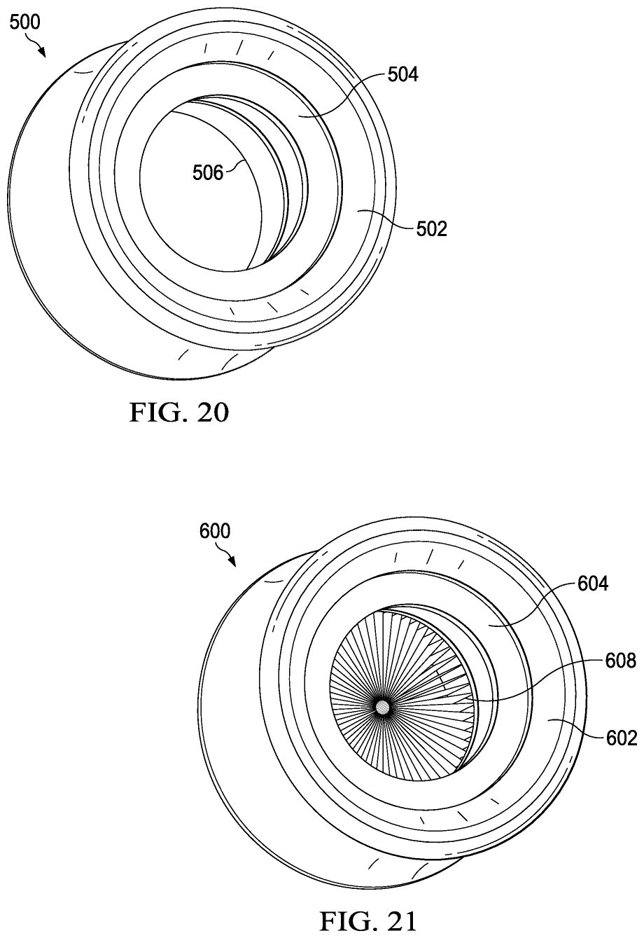

FIGS. 19A-19C and 20 depict a telescoping sleeve implementation that includes an accessory that may be used in conjunction with existing trocar technology to eliminate air leakage during insertion of a stapling instrument into a trocar when the cross-slit valve is opened and shaft of the instrument has not reached the lip seal. As shown in FIGS. 19A-19C, stapling instrument 150 includes handle 152, shaft 154, and end effector 156. As shown in FIG. 20, hub 500 includes retaining ring 502, lip seal 504, and sheath or sleeve 506. In FIG. 19A, hub 500 has not yet been placed on end effector 156. In FIG. 19B, hub 500 has been placed on end effector 156 and sleeve 506 is shown partially extended along the length of end effector 156. In FIG. 19C, sleeve 506 has been fully extended along end effector 156 and has reached shaft 154 of stapling instrument 150. Hub 500 acts as a housing for internal components and also provides an exterior gripping surface for the user to slide sleeve 506 onto stapling instrument 150 and easily translate sleeve 506 along the axis of shaft 154. Lip seal 504 is similar to what is used in existing trocars and is designed to form a seal on the shaft of an instrument being inserted into the trocar. Another important aspect of this component is its drag force on the shaft of the stapling instrument. Lip seal 504 is designed to have less drag relative to the shaft of the instrument compared with the drag between the lip seal of the trocar and the sleeve. Retaining ring 502 houses and retains lip seal 504, an angled cap, and flared end of sleeve 506. Retaining ring 502 may be attached to hub 500 using ultrasonic welding, adhesives, a snap fit, or any other suitable attachment means. Retaining ring 502 provides enough axial preload on the components contained therein to create a hermetic seal that does not allow air leakage during use. Sleeve 506 is typically expandable thin wall rubber, low-density polyethylene (LDPE), or similar material which resides within hub 500 and includes two or more sections. Inserting a stapling instrument into the hub extends each section of sleeve 506 similar to a telescope so that the entire length of the stapling jaws is sealed.

Alternative implementations of the telescoping sleeve include a locking feature on hub 500 that locks the hub onto the proximal end of handle 152 when stapling instrument 150 is fully inserted into sleeve 506. This locking feature may include a quick-connect fitting, a radial compression spring that locks onto shaft 154, threads on hub or sleeve 506 that snap past prongs/flanges in the shrouds of handle 152, an extrusion on the handle shrouds over which a tube slides, an undercut on the handle shrouds that a tube slides into, and/or grooves on an outer tube that hub 500 on sleeve 506 snaps into. Additional variations include a locking feature on hub 500 that locks onto the proximal end of a trocar.

FIG. 21 depicts a bristle valve implementation that includes an accessory that may be used in conjunction with existing trocar technology to eliminate air leakage during insertion of a stapling instrument into a trocar when the cross-slit valve is opened and shaft of the instrument has not reached the lip seal. Hub 600 acts as a housing for internal components and also provides an exterior gripping surface for the user to slide a sleeve onto a stapling instrument and easily translate the sleeve (e.g., sleeve 506) along the axis of the shaft of a stapling instrument. Lip seal 604 is similar to what is used in existing trocars and is designed to form a seal on the shaft of an instrument being inserted into the trocar. Another important aspect of this component is its drag force on the shaft of the stapling instrument. Lip seal 604 is designed to have less drag relative to the shaft of the instrument compared with the drag between the lip seal of the trocar and the sleeve. Retaining ring 602 houses and retains lip seal 604, an angled cap, and flared end of the sleeve. Retaining ring 602 may be attached to hub 600 using ultrasonic welding, adhesives, a snap fit, or any other suitable attachment means. Retaining ring 602 provides enough axial preload on the components contained therein to create a hermetic seal that does not allow air leakage during use. Bristles 608 line the inside of hub 600 in a circular pattern. When a stapling instrument is inserted into hub 600, bristles 608 fill any gap between the upper and lower jaws of the end effector component of the stapling instrument, which reduces the rate of any insufflation leaks as the stapling instrument is inserted through a trocar.

Alternative implementations of the bristle valve include a locking feature on hub 600 that locks the hub onto the proximal end of the handle of a stapling instrument when the instrument is fully inserted into the sleeve. This locking feature may include a quick-connect fitting, a radial compression spring that locks onto the shaft of the instrument, threads on the sleeve that snap past prongs/flanges in the shrouds of the handle, an extrusion on the handle shrouds over which a tube slides, an undercut on the handle shrouds that a tube slides into, and/or grooves on an outer tube that hub 600 on the sleeve snaps into. Additional variations include a locking feature on hub 560 that locks onto the proximal end of a trocar.

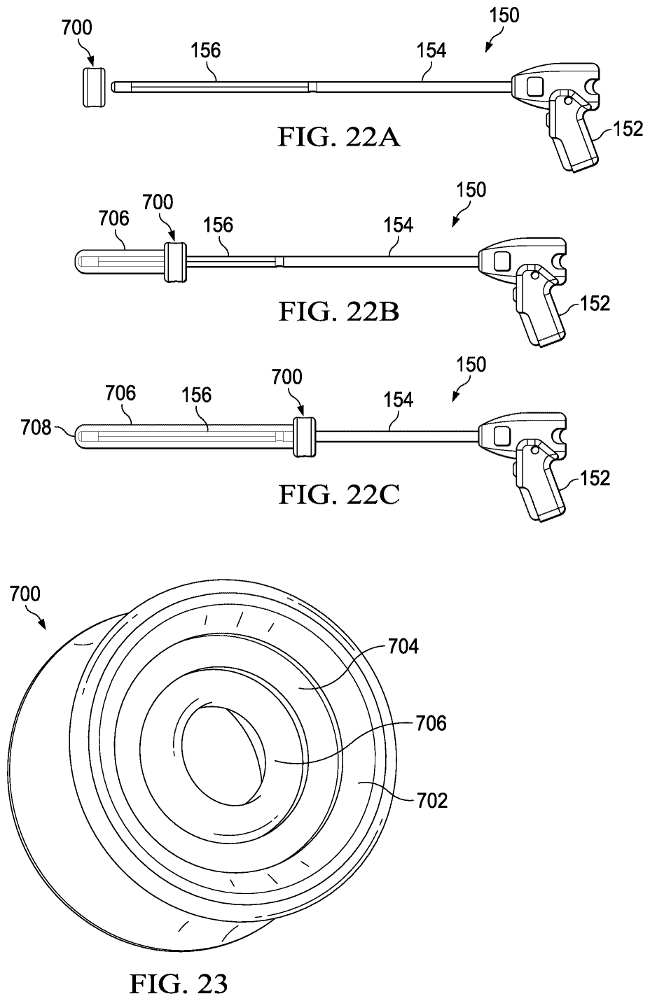

FIGS. 22A-22C and 23 depict a rolled sheath implementation that includes an accessory that may be used in conjunction with existing trocar technology to eliminate air leakage during insertion of a stapling instrument into a trocar when the cross-slit valve is opened and shaft of the instrument has not reached the lip seal. As shown in FIGS. 22A-22C, stapling instrument 150 includes handle 152, shaft 154, and end effector 156. As shown in FIG. 23, hub 700 includes retaining ring 702, lip seal 704, and fully rolled sheath 706. In FIG. 22A, hub 700 has not yet been placed on end effector 156. In FIG. 19B, hub 700 has been placed on end effector 156 and sheath 706 is shown partially unrolled along the length of end effector 156. In FIG. 19C, sheath 706 has been fully unrolled along end effector 156 and has reached shaft 154 of stapling instrument 150. Hub 700 acts as a housing for internal components and also provides an exterior gripping surface for the user to slide sheath 706 onto stapling instrument 150 and easily translate sheath 706 along the axis of shaft 154. Lip seal 704 is similar to what is used in existing trocars and is designed to form a seal on the shaft of an instrument being inserted into the trocar. Another important aspect of this component is its drag force on the shaft of the stapling instrument. Lip seal 704 is designed to have less drag relative to the shaft of the instrument compared with the drag between the lip seal of the trocar and the sheath. Retaining ring 702 houses and retains lip seal 704, an angled cap, and flared end of sheath 706. Retaining ring 702 may be attached to hub 700 using ultrasonic welding, adhesives, a snap fit, or any other suitable attachment means. Retaining ring 702 provides enough axial preload on the components contained therein to create a hermetic seal that does not allow air leakage during use. Sheath 706 is typically expandable thin wall rubber, low-density polyethylene (LDPE), or similar material which resides within hub 700. Inserting stapling instrument 150 into hub 700 unrolls sheath 706 as the instrument is inserted such that the entire length of the jaws of end effector 156 is sealed when the instrument is fully inserted. An aperture 708 formed in the distal end of sheath 706 allows an instrument to be pushed therethrough by expanding around the end-effector.

Alternative implementations of the rolled sheath include a locking feature on hub 700 that locks the hub onto the proximal end of handle 152 when stapling instrument 150 is fully inserted into sheath 706. This locking feature may include a quick-connect fitting, a radial compression spring that locks onto shaft 154, threads on sheath 706 that snap past prongs/flanges in the shrouds of handle 152, an extrusion on the handle shrouds over which a tube slides, an undercut on the handle shrouds that a tube slides into, and/or grooves on an outer tube that hub 700 on sheath 706 snaps into. Additional variations include a locking feature on hub 700 that locks onto the proximal end of a trocar.

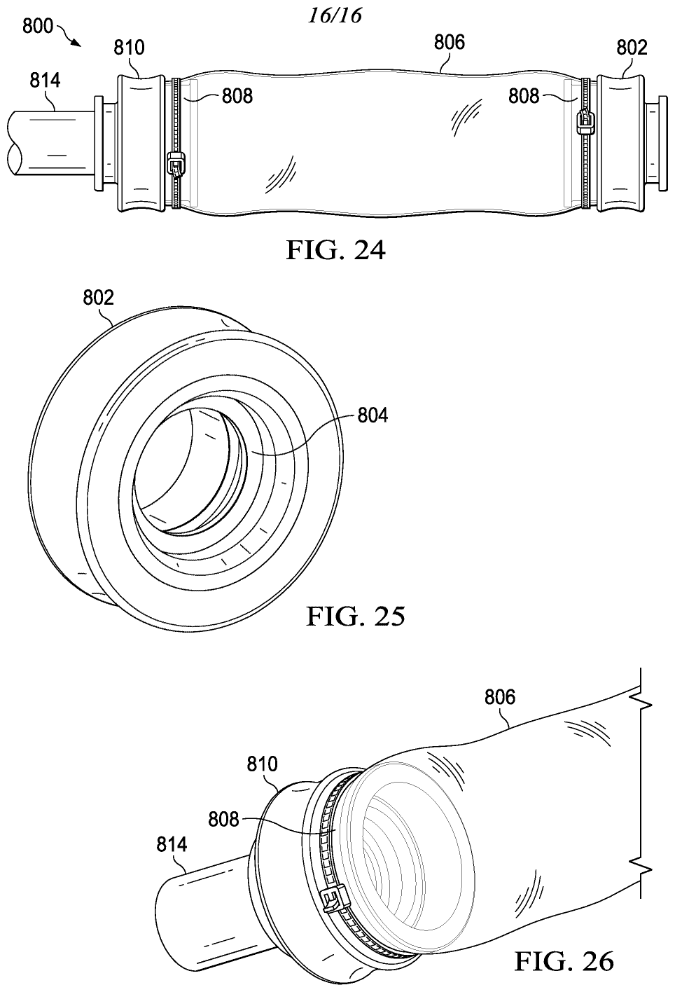

FIGS. 24-26 depict a collapsible sleeve assembly implementation that includes an accessory that may be used in conjunction with existing trocar technology to eliminate air leakage during insertion of a stapling instrument into a trocar when the cross-slit valve is opened and shaft of the instrument has not reached the lip seal. As best shown in FIG. 24, collapsible sleeve assembly 800 includes proximal hub 802 which houses lip seal 804, sleeve 806, sleeve mating structures 808, trocar mating hub 810 which houses a lip seal, and sheath 814 attached thereto. Proximal hub 802 acts as a housing for internal components and also provides an exterior gripping surface for the user to slide sleeve 806 onto a stapling instrument and easily translate sleeve 806 along the axis of the shaft of a stapling instrument. Lip seal 804 is similar to what is used in existing trocars and is designed to form a seal on the shaft of an instrument being inserted into the trocar. Another important aspect of this component is its drag force on the shaft of the stapling instrument. Sleeve 806 is a thin walled bag made from low-density polyethylene (LDPE) or similar material that is thin enough to collapse when compressed and to expand without ripping when pulled under tension. Trocar mating hub 810 acts as a housing in which to mount internal components and also provides an exterior gripping surface for the user when sliding collapsible sleeve 800 into a trocar. Trocar mating hub 810 remains flush with the trocar when inserted into the trocar. Sleeve mating structures 808 cooperate with sleeve 806 to create an airtight seal around trocar mating hub 810 and proximal hub 802. Sheath 814 is a thin walled tube made from high-density polyethylene (HDPE) or similar material that is rigid enough to not collapse during use while thin enough to not significantly increase the internal diameter of the trocar. In an example implementation, the wall thickness of the sheath is about 0.008″ thick. Sheath 814 is designed to have more drag relative to the trocar lip seal than the drag between lip seal 804 in trocar mating hub 810 and the stapling instrument.

Alternative implementations of the collapsible sleeve assembly include a locking feature on proximal hub 802 that locks the hub onto the proximal end of the handle of a stapling instrument when the instrument is fully inserted into sleeve 806. This locking feature may include a quick-connect fitting, a radial compression spring that locks onto the shaft of the instrument, threads on sleeve 806 that snap past prongs/flanges in the shrouds of the handle, an extrusion on the handle shrouds over which a tube slides, an undercut on the handle shrouds that a tube slides into, and/or grooves on an outer tube that proximal hub 802 on sleeve 806 snaps into.

As previously stated and as used herein, the singular forms “a,” “an,” and “the,” refer to both the singular as well as plural, unless the context clearly indicates otherwise. The term “comprising” as used herein is synonymous with “including,” “containing,” or “characterized by,” and is inclusive or open-ended and does not exclude additional, unrecited elements or method steps. Although many methods and materials similar or equivalent to those described herein can be used, particular suitable methods and materials are described herein. Unless context indicates otherwise, the recitations of numerical ranges by endpoints include all numbers subsumed within that range. Furthermore, references to “one implementation” are not intended to be interpreted as excluding the existence of additional implementations that also incorporate the recited features. Moreover, unless explicitly stated to the contrary, implementations “comprising” or “having” an element or a plurality of elements having a particular property may include additional elements whether or not they have that property.

The terms “substantially” and “about” used throughout this specification are used to describe and account for small fluctuations, such as due to variations in processing. For example, these terms can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%, and/or 0%.

There may be many alternate ways to implement the disclosed inventive subject matter. Various functions and elements described herein may be partitioned differently from those shown without departing from the scope of the disclosed inventive subject matter. Generic principles defined herein may be applied to other implementations. Different numbers of a given module or unit may be employed, a different type or types of a given module or unit may be employed, a given module or unit may be added, or a given module or unit may be omitted.

While several devices and components thereof have been discussed in detail above, it should be understood that the components, features, configurations, and methods of using the devices discussed are not limited to the contexts provided above. In particular, components, features, configurations, and methods of use described in the context of one of the devices may be incorporated into any of the other devices. Furthermore, not limited to the further description provided below, additional and alternative suitable components, features, configurations, and methods of using the devices, as well as various ways in which the teachings herein may be combined and interchanged, will be apparent to those of ordinary skill in the art in view of the teachings herein.

Versions of the devices described above may be actuated mechanically or electromechanically (e.g., using one or more electrical motors, solenoids, etc.). However, other actuation modes may be suitable as well including but not limited to pneumatic and/or hydraulic actuation, etc. Various suitable ways in which such alternative forms of actuation may be provided in a device as described above will be apparent to those of ordinary skill in the art in view of the teachings herein.

Versions of the devices described above may have various types of construction. By way of example only, any of the devices described herein, or components thereof, may be constructed from a variety of metal and/or plastic materials.

Having shown and described various versions in the present disclosure, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

What is claimed is:

1. A trocar sheath comprising:
a sheath cannula defining an interior lumen having an open distal end and an open proximal end; and
a housing assembly comprising a sheath hub coupled to the open proximal end of the sheath cannula;
wherein the trocar sheath is slidably and removably extendable within a trocar.

2. The trocar sheath of claim 1, wherein the trocar comprises a trocar seal assembly and, when the trocar sheath is positioned in the trocar, the trocar seal assembly cooperates with the trocar sheath to sealingly engage an outer surface of the trocar sheath.

3. The trocar sheath of claim 1, wherein the housing assembly further comprises a sheath seal contained in the sheath hub, and wherein a first drag on an instrument being inserted through the sheath seal is less than a second drag on the instrument being removed through the sheath seal.

4. The trocar sheath of claim 1, wherein the sheath cannula extends distally of a distal end of the trocar.

5. The trocar sheath of claim 1, wherein, when an instrument is positioned in the trocar sheath, the housing assembly of the trocar sheath cooperates with the instrument to sealingly engage an outer surface of the instrument.

6. The trocar sheath of claim 1, wherein the housing assembly further comprises a seal plate positioned in the sheath hub and coupled to an interior of the open proximal end of the sheath cannula.

7. The trocar sheath of claim 6, wherein the open proximal end of the sheath cannula has an outwardly flared portion that corresponds to a flared portion of the seal plate.

8. The trocar sheath of claim 1, wherein the sheath hub is coupled to an exterior of the open proximal end of the sheath cannula.

9. The trocar sheath of claim 3, wherein the sheath seal is a lip seal.

10. The trocar sheath of claim 3, wherein the housing assembly further comprises a sheath cap contained in the sheath hub proximal of the sheath seal.

11. The trocar sheath of claim 10, wherein the sheath cap is configured to provide an axial preload on the open proximal end of the sheath cannula and the sheath seal to provide a hermetic seal.

12. The trocar sheath of claim 1, wherein a thickness of the sheath cannula is in a range of 0.005 inches to 0.01 inches.

13. A trocar assembly comprising the trocar sheath and the trocar of claim 1, wherein the trocar sheath is positioned in the trocar.

14. The trocar assembly of claim 13, wherein the trocar assembly is a retrofit trocar assembly.

15. A kit comprising the trocar sheath and the trocar of claim 1, wherein the trocar comprises a trocar cannula, a trocar housing, and a trocar seal assembly.

16. The kit of claim 15, further comprising an obturator comprising a handle and a distal blade, wherein the obturator is slidably and removably extendable within the trocar.

17. The kit of claim 15, further comprising an adapter configured to be inserted into an opening of the trocar housing, the adaptor having an opening smaller than the opening of the trocar housing.

18. The kit of claim 17, wherein the adapter includes an adapter housing and an adapter cannula and, when the adapter is inserted in the trocar, the adapter housing abuts the trocar housing, and a distal end of the adapter cannula extends into and is fluidically coupled with the trocar cannula.

19. A method for reducing insufflation loss due to gas leakage during a laparoscopic surgical procedure, wherein the laparoscopic surgical procedure includes the use of a a trocar through which an end effector of a surgical instrument is inserted for access to an abdominal cavity of a patient, the trocar comprising a lip, a lip seal disposed within the lip, and a cannula located distal of the lip, the method comprising providing a sealing device, wherein the sealing device is
(a) disposed within the cannula of the trocar,
(b) configured as an accessory to the trocar, or
(c) integrated into the surgical instrument,
wherein the sealing device is disposed within the cannula of the trocar and includes a diametrically expanding valve,
wherein the diametrically expanding valve is a cross-slit valve, dilating valve, or trap door valve.

20. The method of claim 19, further comprising providing a sealing valve disposed within the cannula, wherein the sealing device is disposed within the cannula of the trocar and includes a balloon, and wherein the balloon is attached to a balloon pump tube that is operative to inflate or deflate the balloon.
